(19) United States
(12) Patent Application Publication
(10) Pub. No.: US 2008/0119833 A1
(43) Pub. Date: May 22, 2008

(54) Cryoprobe with Heating and Temperature Sensing Capabilities

(76) Inventors: David W. Vancelette, San Diego, CA (US); Randall C. Lieser, Plymouth, MN (US)

Correspondence Address: AMS RESEARCH CORPORATION, 10700 BREN ROAD WEST, MINNETONKA, MN, 55343, US

(21) Appl. No.: 11/940,485

(22) Filed: Nov. 15, 2007

Related U.S. Application Data
(60) Provisional application No. 60/866,238, filed on Nov. 17, 2006.

(51) Int. Cl. A61B 18/02

(52) U.S. Cl. 606/20

(57) ABSTRACT

The present disclosure is directed to a cryoprobe including heating capabilities within the cryoprobe tip for use in a cryosurgical system. A heating element can be operably secured to an inner surface of the cryoprobe tips, wherein the heating element can then be connected to an electrical current source such that heat is generated at the cryoprobe tip as the electrical current flows through the heating element. In one version, the heating element can comprise a resistive element laminated between layers of insulation while in other, alternative versions, the heating element can comprise a small diameter resistance wire attached directly to an inner surface within the cryoprobe tip. The cryoprobe can include a thermocouple secured within the cryoprobe tip so as to take temperature measurements during both freezing and thawing cycles. In some versions, the heating element can be operably secured within the cryoprobe tip using an expanding or rotating mandrel.

### PRIORITY CLAIM

The present application claims priority to U.S. Provisional Application Ser. No. 60/866,238, filed Nov. 17, 2006 and entitled “CRYOPROBE WITH HEATING AND TEMPERATURE SENSING CAPABILITIES”, which is herein incorporated by reference in its entirety.

### FIELD OF THE INVENTION

The present disclosure relates to cryoprobes for use in cryosurgical systems for treatment of benign or cancerous tissues. More particularly, the present invention pertains to cryoprobes and related methods of constructing the cryoprobes to incorporate electrical heating and thermal sensing capabilities.

### BACKGROUND OF THE INVENTION

Cryosurgical probes are used to treat a variety of diseases. Cryosurgical probes quickly freeze diseased body tissue, causing the tissue to die after which it will be absorbed by the body, expelled by the body, sloughed off or replaced by scar tissue. Cryothermal treatment can be used to treat prostate cancer and benign prostate disease. Cryosurgery also has gynecological applications. In addition, cryosurgery may be used for the treatment of a number of other diseases and conditions including, but certainly not limited to, breast cancer, liver cancer, renal cancer, glaucoma and other eye diseases.

A variety of cryosurgical instruments variously referred to as cryoprobes, cryosurgical probes, cryosurgical ablation devices, cryostats and cryocoolers have been used for cryosurgery. These devices typically use the principle of Joule-Thomson expansion to generate cooling. They take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue. A properly performed cryosurgical procedure allows cryoablation of the diseased tissue without undue destruction of surrounding healthy tissue.

Cryosurgery often involves a cycle of treatments in which the targeted tissue is frozen, allowed to thaw, and then refrozen. Thawing can occur naturally or can be accelerated by use of a heat source. Double and even triple freeze/thaw cycles are now commonly used in cryosurgery. When comparing a single freeze/thaw cycle with treatment regimens involving multiple freeze/thaw cycles, it has been observed that the additional freeze/thaw cycles can lead to an increase the damage/destruction of the targeted tissue, thus providing for a more beneficial and efficacious treatment.

### SUMMARY OF THE INVENTION

The present disclosure is directed to a cryoprobe including heating capabilities within the cryoprobe tip for use in a cryosurgical system. A heating element can be operably secured to an inner surface of the cryoprobe tips, wherein the heating element can then be connected to an electrical current source such that heat is generated at the cryoprobe tip as the electrical current flows through the heating element. In some embodiments, the heating element can comprise a resistive element laminated between layers of insulation while in other, alternative embodiments, the heating element can comprise a small diameter resistance wire attached directly to an inner surface within the cryoprobe tip. In some embodiments, a thermocouple can be secured within the cryoprobe tip so as to take temperature measurements during both freezing and thawing cycles. In some embodiments, the heating element can be operably secured within the cryoprobe tip using an expanding or rotating mandrel.

In one aspect of the present disclosure, a cryoprobe for use in a cryosurgical system includes a resistive heating element. The heating element can be secured to an inner surface of a cryoprobe tip and subsequently connected to an electrical current source. A thermocouple can be secured in combination with the heating element so as to measure temperature during freezing and thawing cycles. In some embodiments, the thermocouple can be operably connected to a thermal cutoff so as to break the electrical circuit between the electrical current source and the heating element if the cryoprobe tip exceeds a selected temperature. In some embodiments, the cryoprobe can further include a Joule-Thompson expansion element and related fluid channels so that it can alternately be used for both freezing and heating.

In another aspect of the present disclosure, representative methods for securing resistive heating elements within a cryoprobe tip can include the use of an expandable mandrel. In one embodiment, the expandable mandrel can include a substantially cylindrical body having a plurality of longitudinal grooves and a rounded end that can conform to the inner geometry of a cryoprobe tip. The expandable mandrel can further include a connector for connecting to a pneumatic pressure source. Alternatively, the expandable mandrel can comprise a pair of substantially half-cylinder portions having longitudinal grooves and a rounded end. The half-cylinder portions can create an opening extending longitudinally through the mandrel.

In yet another aspect of the present disclosure, an expandable mandrel can be used to secure heating elements to the inner surfaces of cryoprobe tips. Heating elements can be positioned within longitudinal grooves an on outer surface of the expandable mandrel and coated with an adhesive. The expandable mandrel can then be inserted into the cryoprobe tip and expanded to press the heating elements against the inner surface of the cryoprobe tip until the adhesive cures. In one representative embodiment, the expandable mandrel can comprise a flexible material capable of being expanded using pneumatic pressure. In another representative embodiment, the expandable mandrel can comprise a pair of body members capable of being outwardly biased by an insertion pin that is rotated though a center opening defined between the body members. Once the heating elements are secured to the inner surface of the cryoprobe tip, the biasing means can be removed such that the expandable mandrel can be removed.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a side view of an embodiment of a representative cryosurgical system in which cryoprobes of the present disclosure may be used.

FIG. 2 is a side, section view of a cryoprobe tip according to an embodiment of a cryoprobe of the present disclosure.

FIG. 3 is a perspective, end view of an embodiment of an expandable mandrel for attaching heating elements to the inside of the cryoprobe tip of FIG. 2.

FIG. 4 is a perspective, end view of an embodiment of an expandable mandrel for attaching heating elements to the inside of the cryoprobe tip of FIG. 2.

### DETAILED DESCRIPTION

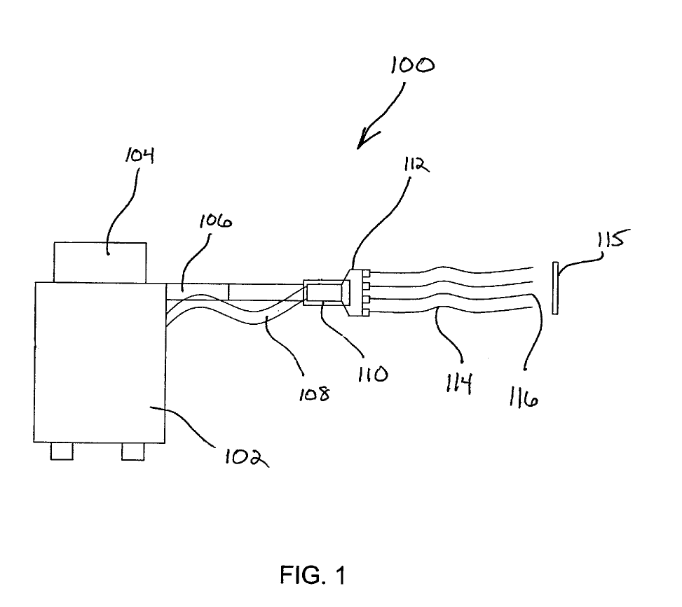

A representative closed loop cryosurgical system 100 that can be used with cryoprobes according to the present disclosure is illustrated in FIG. 1. Cryosurgical system 100 can include a refrigeration and control console 102 with an attached display 104. Control console 102 can contain a primary compressor to provide a primary pressurized, mixed gas refrigerant to the system and a secondary compressor to provide a secondary pressurized, mixed gas refrigerant to the system. The use of mixed gas refrigerants is generally known in the art to provide a dramatic increase in cooling performance over the use of a single gas refrigerant. Control console 102 can also include controls that allow for the activation, deactivation, and modification of various system parameters, such as, for example, gas flow rates, pressures, and temperatures of the mixed gas refrigerants. Display 104 can provide the operator the ability to monitor, and in some embodiments adjust, the system to ensure it is performing properly and can provide real-time display as well as recording and historical displays of system parameters. One exemplary console that can be used with an embodiment of the present invention is used as part of the Her Option® Office Cryoablation Therapy available from American Medical Systems of Minnetonka, Minn.

With reference to FIG. 1, the high pressure primary refrigerant is transferred from control console 102 to a cryostat heat exchanger module 110 through a flexible line 108. The cryostat heat exchanger module 110 can include a manifold portion 112 that transfers the refrigerant into and receives refrigerant out of one or more cryoprobes 114. The cryostat heat exchanger module 110 and cryoprobes 114 can also be connected to the control console 102 by way of an articulating arm 106, which may be manually or automatically used to position the cryostat heat exchanger module 110 and cryoprobes 114. Although depicted as having the flexible line 108 as a separate component from the articulating arm 106, cryosurgical system 100 can incorporate the flexible line 108 within the articulating arm 106. A positioning grid 115 can be used to properly align and position the cryoprobes 114 for patient insertion.

Each cryoprobe 114 has a tip 116 that constitutes the region of the cryoprobe 114 that performs the actual cryogenic treatment. The tip 116 contains the Joule-Thompson expansion element 119, such as a capillary tube, through which refrigerant can be expanded to create the cold temperatures used to freeze diseased tissue. During a cooling cycle, an iceball is formed at tip 116 that is subsequently positioned against diseased tissue such that tissue is frozen and dies.

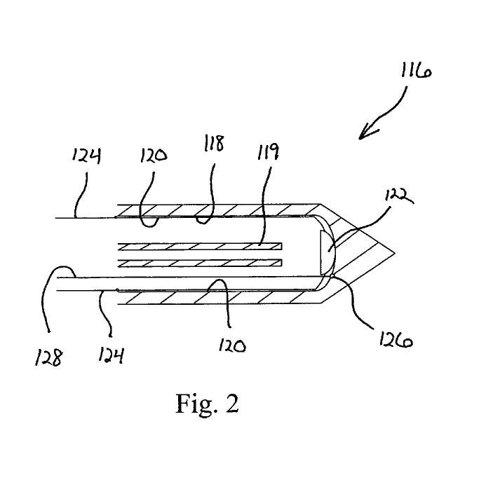

As presently contemplated, cryoprobe 114 can also contain electrical heating and/or thermal sensing elements within tip 116, as illustrated in FIG. 2. As such, cryoprobes of the present disclosure can be used for conducting sequential freezing and thawing cycles. Cryoprobe tip 116 can include one or more heating elements 120 adhered to an inner surface 118 of tip 116. Heating elements 120 can include leads 124 for operable connection to an electrical current source. In some presently preferred embodiments, the electrical current source can be integral to and located within the control console 102. Heating elements 120 can also connect to a thermal cutoff 122, which can also be secured to the inner surface 118 of cryoprobe tip 116. If the temperature in the system is increased to an abnormal or unsafe level, the thermal cutoff 122 senses the change and breaks the electrical circuit. A thermocouple 126 can also be included to transmit temperature measurements at tip 116 through a lead 128 to the control console 102 and display 104. The thermocouple can wrap with the heating elements 120 or can attach to the inner surface 118 of the cryoprobe tip 116 and “float” inside the tip 116.

Various heating elements 120 can be used with cryoprobes 114. One representative heating element 120 can comprise a wrapped thermofoil heater having an etched foil, resistive element laminated between two layers of flexible, thin insulation. Such a heating element can encircle the full inner circumference of the cryoprobe tip 116 or only partly cover it by attaching a strip to one “side” of the inner surface 118 of the cryoprobe tip 116. Alternatively, heating element 120 can comprise a small diameter (0.003 in. to 0.008 in.) resistance wire. Resistance wire can run in a longitudinal direction along the inner surface 118 of cryoprobe tip 116 with 180 degree loops at either end of the cryoprobe tip 116. Where desired, the amount of wire can be increased by attaching lengths of wire at different radial points around the circumference of the cryoprobe tip 116.

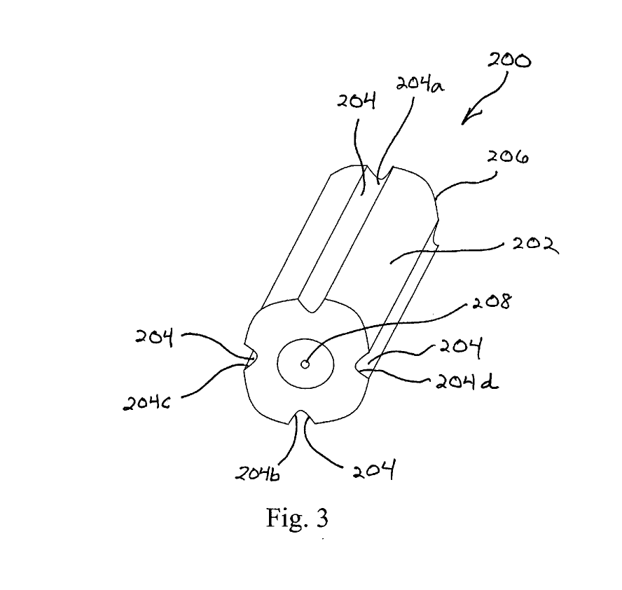

Because of the small inside diameter of cryoprobe tips 116 (typically 1.5-2.5 mm), it can be difficult to secure heating elements to the inner surface 118 of tips 116. In one presently contemplated fabrication method, heating elements 120 can be secured to the inner surface 118 of cryoprobe tips 116 with an expandable mandrel 200 as illustrated in FIG. 3. Mandrel 200 can comprise a substantially cylindrical body 202 with a plurality of external, longitudinal grooves 204 and a rounded end 206 that can conform to the internal geometry of a cryoprobe tip 116. In some representative embodiments, mandrel 200 can comprise a thin, inflatable material such as silicone. Mandrel 200 can also include a connector 208 that can be operably coupled to a pneumatic pressure source.

A first step in securing heating elements 120 to the inner surface 118 of cryoprobe tips 116 with mandrel 200 involves positioning the heating elements 120 within the external, longitudinal grooves 204. For instance, one heating element 120 can positioned so as to run along the length of a first groove 204a, loop over the rounded end 206 of mandrel 200, and run back along the length of a second groove 204b that is 180 degrees opposed to the first groove 204. A second heating element 120 can be similarly positioned within third groove 204c, looped over rounded end 206 and run back within fourth groove 204d. When looping the heating elements 120 over the rounded end 206, it is preferable that some slack be left at the end of the loop to accommodate expansion of the mandrel within the cryoprobe tip 116 as described below. By looping the heating elements 120 over the rounded end 206, a complete heating circuit can be positioned at the cryoprobe tip 116 to generate heat. The heating elements 120 can then be coated, covered and/or encased in an adhesive selected so as to not bond with the mandrel 200. A mold release compound and/or other lubricant can also be used to ensure that the heating elements 120 do not adhere to the mandrel 200.

Once the heating elements 120 have been positioned, the mandrel 200 can be inserted into the cryoprobe tip 116. A pneumatic pressure source can then be connected to mandrel 200 via connector 208 in order to expand the mandrel 200. A mandrel 200 comprised of a thin, inflatable material will inflate like a balloon until the heating elements 120 are flush with the inner surface of cryoprobe tip 116. Mandrel 200 is left in this inflated disposition within the cryoprobe tip 116 until the adhesive cures. Preferably, the adhesive is somewhat viscous so that it remains within the grooves 204 and does not leak out elsewhere within the cryoprobe 114 before it cures. The mandrel 200 can then be removed and the heating elements 120 will remain secured to the inner surface 118 of cryoprobe tip 116. Mandrel 200 can also be used to secure a thermal cutoff 122 and a thermocouple 126 to the inner surface of cryoprobe tip 116.

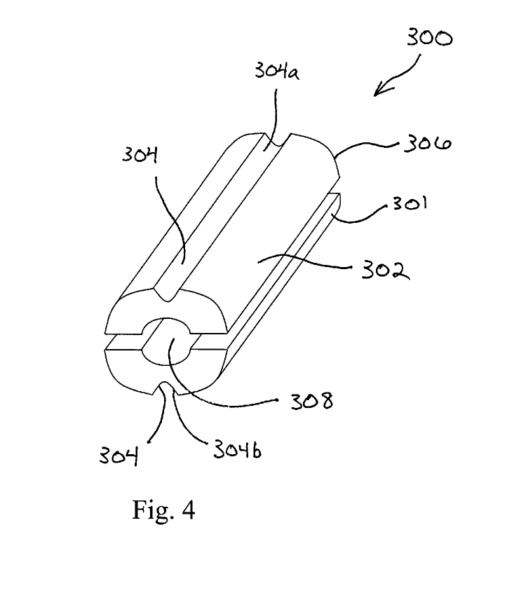

In an alternative attachment step, heating elements 120 can be secured to cryoprobe tip 116 using a mandrel 300 as illustrated in FIG. 4. Mandrel 300 can comprise a pair of substantially half-cylindrically shaped mandrel portions 301, 302 with a plurality of external, longitudinal grooves 304 and a rounded end 306 that conforms to the internal geometry of a cryoprobe tip 116. Mandrel 300 can further include a central opening 308 where mandrel portions 301, 302 rest flush with each other. Mandrel 300 is preferably fabricated of a material that will not bond with an adhesive, such as PolyTetraFluoroEthylene (PTFE).

As with mandrel 200, heating elements 120 can be positioned with respect to mandrel 300 such that the heating elements 120 are run through groove 304a, looped about rounded end 306, through groove 304b and coated with an adhesive. Following insertion of the mandrel 300 within the cryoprobe tip 166, mandrel 300 can be expanded by inserting a pin or other rotatable center piece to force the mandrel portions 301, 302 apart so that the heating elements 120 are held against the inner surface 118 of cryoprobe tip 116. Once the adhesive cures, mandrel 300 can be removed.

As an alternative to a conventional adhesive, an Ultra-Violet (UV) curable epoxy can be used in conjunction with both mandrel 200 and mandrel 300 to securing heating elements 120 to the inner surface 118 of cryoprobe tip 116. When a UV curable epoxy is used, mandrel 200 and mandrel 300 can each be fabricated of a transparent material. Mandrel 200 and mandrel 300 can each include a UV light source contained therein. Upon insertion and expansion of mandrel 200 or mandrel 300, the UV light source can be activated so as to cure the UV epoxy and secure the heating elements 120 to the inner surface 118 of cryoprobe tip 116.

Once the heating elements 120 are secured inside the cryoprobe tip 116, the leads 124, 128 can be connected to the control console 102. Control console 102 can selectively control the flow of electrical current through the heating elements 120 depending upon whether the treatment plan is operating in a freeze or thaw cycle. By using resistive heating elements, the use of heated gases and/or liquids and the associate flow channels necessary for their use can be avoided within the cryosurgical system 100. The use of electric resistive heating elements can also provide for faster and more responsive temperature adjustment and transitions at the cryoprobe tip 116. The thermocouple 126 can be used to measure the tip 116 temperature during both freezing and heating cycles.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

1. A cryoprobe for use in a cryosurgical system, comprising:
a tip portion for placement against selected tissue during a cryosurgical procedure involving at least one freeze cycle and at least one thaw cycle;
an expansion element for expanding a refrigerant in the tip portion to cooled the tip portion during a freeze cycle; and
at least one heating element secured along an inner surface of the tip portion, the at least one heating element having control leads configured to operably connect the at least one heating element to an electrical current source for heating the tip portion during a thaw cycle.

2. The cryoprobe of claim 1, further comprising a thermal sensing element secured with tip portion to provide temperature measurements at the tip portion to a control console.

3. The cryoprobe of claim 2, wherein the thermal sensing element is secured to the at least one heating element.

4. The cryoprobe of claim 2, wherein the thermocouple is attached to the inner surface of the tip portion.

5. The cryoprobe of claim 1, further comprising a thermal cutoff operably connected to the at least one heating element, the thermal cutoff configured to selectively break the electrical connection between the at least one heating element and the electrical current source if a temperature at the tip portion exceeds a predetermined threshold temperature.

6. The cryoprobe of claim 1, wherein the at least one heating element comprises a thermofoil heater.

7. The cryoprobe of claim 6, wherein the thermofoil heater comprises an etched foil resistive element laminated between two layers of insulation.

8. The cryoprobe of claim 6, wherein the at least one heating element encircles a full inner circumference of the inner surface.

9. The cryoprobe of claim 1, wherein the at least one heating element comprises a resistance wire.

10. The cryoprobe of claim 9, wherein the resistance wire has a diameter between about 0.003 inches and about 0.008 inches.

11. The cryoprobe of claim 9, wherein the heating element runs in a longitudinal direction along the inner surface with a 180° loop at an end of the tip portion.

12. A mandrel for securing heating elements to an inner surface of a cryoprobe tip portion for use in a cryosurgical system comprising:
a substantially cylindrical body adapted to transition between a first non-expanded disposition and a second expanded disposition;
at least two longitudinal channels defined along in the cylindrical body; and
a rounded end adapted to conform to an internal geometry of a cryoprobe tip portion of a cryoprobe.

13. The mandrel of claim 12, wherein the cylindrical body comprises an inflatable material.

14. The mandrel of claim 13, further comprising a connector configured to connect to a pneumatic pressure source for inflating the cylindrical body.

15. The mandrel of claim 12, wherein the cylindrical body comprises a pair of half-cylindrical body portions.

16. The mandrel of claim 15, wherein the pair of half-cylindrical body portions comprises PolyTetraFluoroEthylene.

17. The mandrel of claim 15, further comprising a central opening between the pair of half-cylindrical body portions and a rotatable center piece, said rotatable center piece turning within the cylindrical body to expand the pair of half-cylindrical body portions.

18. A method of securing an item to the inner surface of a cryoprobe for use in a cryosurgical system comprising:
providing a cryoprobe having a tip portion;
providing an expandable mandrel comprising a substantially cylindrical body with a plurality of longitudinal grooves;
positioning a heating element within one or more of the longitudinal grooves;
coating the item with an adhesive, wherein the adhesive comprises a material selected so as to be incompatible with bonding to the expandable mandrel;
inserting the expandable mandrel into the tip portion;
expanding the expandable mandrel until the heating element resides against an inner surface of the tip portion;
curing the adhesive with the expandable mandrel remaining in an expanded position to secure the heating element to the inner surface; and
removing the mandrel from the cryoprobe.

19. The method of claim 18, wherein the expandable mandrel comprises an inflatable material having an inflation connector, and wherein expanding the expandable mandrel comprises:
connecting the inflation connector to a pneumatic pressure source; and
inflating the expandable mandrel by activating the pneumatic pressure source.

20. The method of claim 18, wherein the expandable mandrel comprises a pair of half-cylindrical body portions with a central opening defined between the body portions, and wherein expanding the expandable mandrel comprises:
inserting a center piece into the central opening to force the half-cylindrical body portions apart.
